(19) United States
(12) Patent Application Publication
(10) Pub. No.: US 2009/0275075 A1
(43) Pub. Date: Nov. 5, 2009

(54) METHODS AND SYSTEMS FOR ENSURING THE SECURITY OF FOOD COMMODITIES

(76) Inventors: Eric B. Dodd, Evansville, IN (US); Kevin E. Humphrey, Utica, KY (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/433,542

(22) Filed: Apr. 30, 2009

Related U.S. Application Data
(63) Continuation-in-part of application No. 11/553,163, filed on Oct. 26, 2006.

(51) Int. Cl. C12Q 1/04

(52) U.S. Cl. 435/34

(57) ABSTRACT

Aspects include claims, systems, and methods for testing food commodities, including without limitation bulk food commodity stores to identify the presence of contaminants. Such contaminants tested for may include biological, chemical, or radio nuclear material. Another aspect is marking and/or tracking food commodity stores that have been certified as either being contaminated or safe. Still another aspect is an automated or automatic system for measuring the level of contamination in a given food commodity store, and for marking and tracking food commodity stores tested for contamination.

This application is a continuation of Application Ser. No. 11/553,163, filed Oct. 26, 2006, which is incorporated herein by reference.

### FIELD OF THE INVENTION

Various aspects relate to the testing of food commodities (specifically dry goods including grains, nuts, coffee, tea, etc.) for contamination and/or marking tested commodities and tracking them from various points in the production of food from harvesting to food processors to the food distributers to the retail market and ultimately the consumer.

### BACKGROUND

The production and marketing of dry goods, e.g., grain and grain-related products worldwide is a multi-billion dollar a year industry. In the United States along, about 2.1 million producers deliver about 300 million metric tons of grain to U.S.-based elevators each year and about 1.08 million railroad cars are used to transport grain; in all 23 million metric tons of grain are shipped by barge each year.

In the industrialized world, a vanishing small number of farm-related workers has generated a situation in which most people have very little actual contact with or knowledge of precisely where their food was grown, harvested, shipped, and processed. This also means that most people in industrialized nations live and consume foods far removed from where they are produced. This has led many officials and food safety experts to note how vulnerable the food supply chain system is to either deliberate, natural or inadvertent contamination.

Accordingly, there is a pressing need for methods to enable people to track potential contamination within the food commodities-based food chain. The need to insure a safe supply of food has always existed in the food industry. What has become glaringly apparent of late is that this entire supply chain is vulnerable to nefarious assault as well as natural and other man-made phenomenon.

The threat to the nation's food supply by “Agro-Terrorism” has been detailed in various reports, including, for example, “Terrorism and the Grain Handling System in Canada and the United States,” by William Ngange, William Wilson, and James Nolan. The world-wide threat from Agro-Terrorism has been summarized in a report issued by the United Nations, World Health Organization in a report entitled, “Terrorist Threats to Food: Guidelines for Establishing and Strengthening Prevention and Response Systems.” The World Health Organization defines food terrorism as:

“The act or threat of deliberate contamination of food for human consumption with chemicals, biological and radio nuclear agents for pure reasons of causing injury or death to civilian population and/or disrupting social, economic or political stability.”

These reports and the like focus on assessing the threat that contaminated foods, including, for example, contaminated food commodity stores, transportation and processing, pose to civilian populations. These reports do not propose solutions, although both recommend increased vigilance of the food supply by those responsible for producing and transporting foods, including food commodities including dry goods. Clearly, there is a threat to the world's food supply and there is a need for a means of testing, monitoring, and tracking food commodities including dry goods throughout the food chain system.

Still another concerning for many consumers both in the Untied Sates and worldwide is the intentional or unintended commingling of food commodities which have been genetically modified with a Genetically Modified Organism (GMO). Many consumers expressed a clear preference for varieties of grains for example that are free of GMOs. Many countries and regions exclude or limit the use of GMO food products. Many GMO plants are almost identical to non-GMO plants, differences in some instances being only one or a handful of genes. This makes differentiating between food commodities that are derived from a GMO plant versus non-GMO plant derived claims very difficult. One approach is to carefully document the source of all of the food commodities and to certify the origin of the food-stuff in one practice within the industry such food-stuffs. The current approach does not provide a ready method for widespread easy testing of bulk food commodity stores to empirically certify that the crop is GMO free.

Various aspects disclosed herein address the need for efficient means to test for and determine the presence of various contaminants, including chemical and microbiological agents as well as GMOs in food commodities within the entire food chain.

### SUMMARY

One embodiment is a method for checking the safety of food commodities, including dry goods, comprising the steps of providing a bulk quantity of the food commodity in a container, after harvesting the food commodity and before processing the food commodity into an intermediate or final food product; passing at least a sample of gas-borne material once in contact with a food commodity through a capturing media; extracting at least some of the particulate matter from the capturing media; and testing the matter for the presence of biological contaminants.

One embodiment is a method for checking the safety of food commodities, including dry goods, comprising the steps of providing a bulk quantity of the food commodity in a container, after transporting the food commodity and before intermediate processing of the food commodity into an intermediate food product; passing at least a sample of gas-borne material once in contact with a food commodity through a capturing media; extracting at least some of the particulate matter from the capturing media; and testing the matter for the presence of biological contaminants.

One embodiment is a method for checking the safety of food commodities, including dry goods, comprising the steps of providing a bulk quantity of the food commodity in a container, after intermediate processing of the food commodity and before final processing of the food commodity into an final food product; passing at least a sample of gas-borne material once in contact with a food commodity through a capturing media; extracting at least some of the particulate matter from the capturing media; and testing the matter for the presence of biological contaminants.

One embodiment includes testing the food commodity for biological contaminants selected from the group comprising: bacteria, viruses, molds or gene sequence.

In some embodiments capture media includes a device for recording the time and location at which a given sample was collected, in one embodiment the device is in the form of an RFID tag or bar code and it may incorporate global positioning capability to record the location of the capture media when it was used or the device collected for further analysis. In some aspects the device may be used to transfer data concerning the location of the capture media at the time of sampling to a data base. The data base may also include data concerning subsequent analysis of the sample and these data streams may be mated to provide a history of the sample from capture to analysis. The data may also be used to provide information on the particular food commodity store from which the sample was originally captured.

One embodiment includes a pulsed sampling technique to help ensure that the sample collected in the capture device is representative of the gas in contact with the grain store being sampled.

Stages in food commodity processing and transport in which the food commodity is being moved by air flow (positive or negative pressure) in part or in bulk present attractive opportunities for gather statistically significant samples of materials that were in the food commodity and were associated with gases that are or were in contact with the food commodity.

In one embodiment, the capture media may include at least one of the following: a filter, a static dust collector, a scrubber, a bubble tube, or any combination thereof.

In one embodiment, after a particular grain store has been checked for contamination, the checked batch is marked to identify the contamination status of the batch.

In another embodiment, the checked batch is marked by inserting a Radio-Frequency ID (RFID) device into the food commodity. In another embodiment, the checked batch is marked by a barcode on the food commodity container. In still another embodiment, the store is marked by means of coating, spraying, and/or dyeing the food commodity a particular visible color, or ultra-violet hue, preferably using an food safe material.

In one embodiment, a sample of material collected from the capture media is examined using any of a variety of analytical techniques, including, for example, a Geiger counter, to detect the progress of a radioactive contaminant or any of the following other analytical techniques including, for example, absorbance measurements, fluorescence measurements, antibody binding determinations, polymerase chain reaction, or any combination thereof, or various arrays which may include on a surface or a series of surfaces a number of different materials that specifically interact with various contaminants. Materials that interact with contaminates may include, for example, antibodies, each sensitive to a specific antigen, molecular probes or oligonucleotides that selectively hybridize to specific components of various contaminants.

In one embodiment, a sample collected from a capture media is marked with a unique code, and this code is used to track that particular sample, and by inference the food commodity store from which that sample was collected, throughout the rest of the food chain.

In one embodiment, the food commodity store is monitored within a given food commodity processing facility. In still another embodiment, the food commodity store may be monitored from place to place up through, and including, actual delivery to a processing plant in which a particular food commodity is converted into an intermediate or final food product.

In one embodiment, an automated system is used to alert either human or computerized monitors to the fact that a batch of food commodity has tested positive for contamination or a specific gene sequence which is either present or not present. In one embodiment, contaminated stores are flagged and tracked, for either removal from the system or decontamination. In still another embodiment, information that a store of food commodity is contaminated generates an alert that may be any form including a siren, strobe, e-mail, telephone call, facsimile transfer, or any other means of drawing attention to the fact that a particular sample has tested positive for a given contaminant.

Various sampling devices are envisioned within various embodiments, these supply devices include sampling a given volume of gas in contact with a food commodity store, in order to create a sample that is representative of the content of a particular food commodity store. In some embodiments, the length of time over which particular samples are collected and tested can vary according to the needs of a particular operation and the capacity of the sampling system.

### BRIEF DESCRIPTION OF THE DRAWINGS

FIG. 1 is a block diagram illustrating various steps in one embodiment.

FIG. 2 is still another block diagram illustrating various steps in one embodiment.

FIG. 3 is a schematic representation of various steps in the process of growing and bringing to the retail market grain and grain-related products.

FIG. 4 is a schematic representation of an embodiment involving sampling a grain store, preparing the sample for analysis, analyzing the sample, and generating a report including data gathered on the sample.

FIG. 5 is a hypothetical representation of the typical report which could be generated according to various embodiments.

FIG. 6 is a schematic representation of various types of data related to the content of a grain store that can be collected in conjunction with various embodiments.

FIG. 7 is a block diagram illustrating various steps in an embodiment.

FIG. 8 is still another block diagram illustrating various steps in an embodiment.

### DETAILED DESCRIPTION

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described processes, systems or devices, and any further applications of the principles of the invention as described herein, are contemplated as would normally occur to one skilled in the art to which the invention relates.

Definitions

Terms used herein are given their usual and customary definitions unless stated otherwise.

GRAIN STORE: Any facility or structure used to store harvested grain in bulk. (Example: a grain bin, flat storage, storage tank, a barge, a railroad car, or a truck used for the transportation of grain.) A grain store may be on farm storage, a commercial elevator or bulk grain held by an end user. The term may also refer to the bulk storage tank on a grain harvester “combine”.
GRAIN ELEVATOR: A common term used for a commercial facility that stores bulk grain. The term is sometimes used to describe a piece of equipment used to elevate grain (see ELEVATOR).
ELEVATOR: A piece of equipment used to elevate grain from ground level into a grain storage structure. Common terms for an elevator may include an elevator leg, leg, or grain elevator.
FLAT STORAGE: A common term used for describing the storage of grain on a flat surface such as a building floor or the ground.
STORAGE TANK: A structure used for storing grain, such as a food commodity bin, a bulk tank, etc.
CONVEYOR: A piece of equipment generally consisting of a chain or belt that is used to transfer food commodity to or from storage.
AUGER: A piece of equipment that generally consists of a round tube containing a screw or flight that is used to transfer food commodity to or from storage.
COMBINE: A piece of equipment used to harvest grain. Generally self propelled and containing a bulk storage bin for grain.
GRAIN DRYER: A bulk storage bin built using perforated sides so that heated air may pass through the grain to reduce the moisture content.
BATCH: One food commodity store unit. (Example: barge, railcar, truck, ship hold, or food commodity silo.)
COMPOSITE: Uniform mixture of strategically collected grab samples.
GRAB SAMPLE: A sample collected from a specific location within a specified time of a specific amount (all determined on statistical requirement).
BULK: In large, industrial and/or commercial agricultural quantities, typically more than a cubic meter collectively. Bulk includes food in storage containers, as well as in transportation vehicles, as well as moving along conveyors, augers and conduits, and the like.
FOOD COMMODITY: Any crop, grain, nut or human consumable food (including partially or intermediate processed bulk food (flour, cereals, meals, or otherwise), as well as combinations and/or blends thereof.
DRY GOODS: A food commodity that is granular and is not in a slurry. Preferably, but not necessarily, dry goods have been actively dried by evaporation, forced air and/or heat, but also they may include some moisture. Dry good may include grain, coffee, nuts, etc., and also may include intermediate food commodities (e.g. grain to flour), as well as combinations and/or blends thereof.
MICROBIOLOGICAL COMTAMINANT: Any bacteria, virus or mold which is either natural or non-natural, and which may be adverse to human health. A microbiological contaminant is also inclusive of any material which has been genetically modified and is not naturally occurring and which may be adverse to human health.
HEADSPACE: The gas (usually air) which is surrounding and between the food commodity that may be also referred to as the interstitial space in a bulk of food commodity.

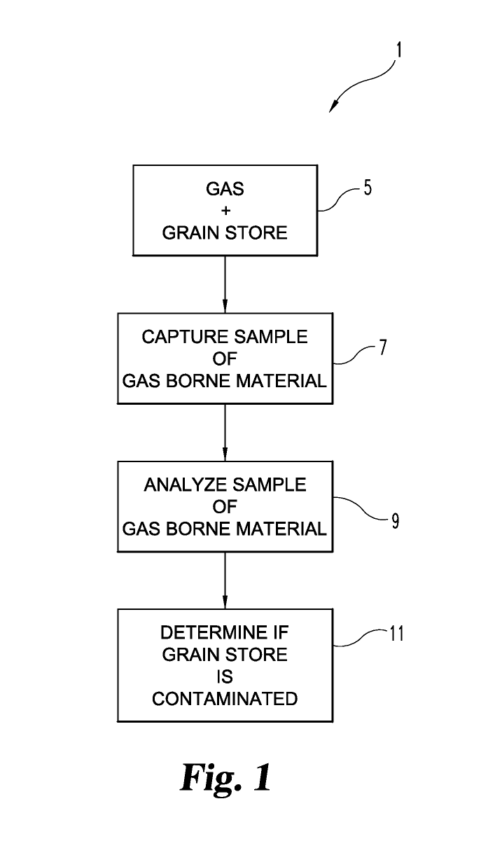

There is a great need for an economical and effective means of testing food commodities at various stages of its harvest, transport, and processing to determine and verify that a given shipment of food commodity is contaminant free when it is delivered to an intermediate or final food processing plant. Various embodiments are directed towards meeting this need. Referring now to FIG. 1, block diagram 1 illustrates various steps in one embodiment. These steps include, but are not limited to, providing a food commodity store which includes a gas 5, generally air. In one aspect, the gas is located in the headspace of a device or structure used for storing food commodity in bulk, for example, a food commodity elevator, storage tank, railroad car, closed truck, shiphold, or the like. Another step illustrated in FIG. 1 is sampling gas that includes gas-borne materials; for example, material including particulates associated with the food commodity are captured on capture media 7. Next, the gas-borne material collected from the captured media 2 is analyzed 9. In still another step of this embodiment, a determination is made as to whether or not a particular food commodity store is contaminated 11.

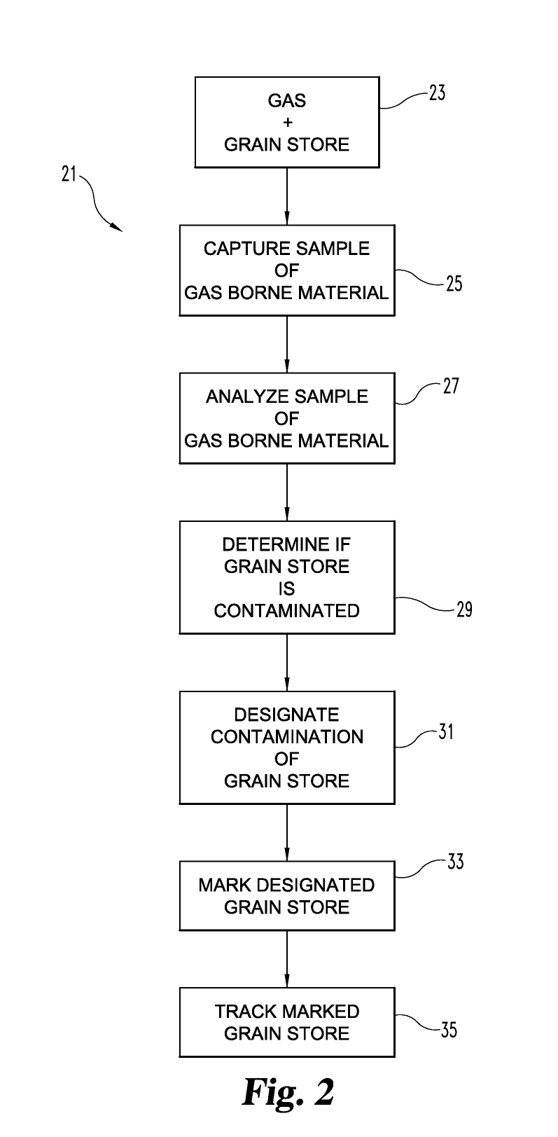

Referring now to FIG. 2, block diagram 21 illustrates various steps in some embodiments. Steps included in these embodiments comprise providing a food commodity store having a gas associated with it that is or was in contact with at least a portion of the grain store 23. Another step includes capturing a sample of the gas-borne material 25, preferably a sample from a statistically significant sample of gas. Another step of this embodiment is analyzing the sample of gas-borne material collected 27 from the gas sample. Next, the results of the analysis performed in step 27 are used to determine if the food commodity store is contaminated 29. Based on the determination made in step 29, a food commodity store may be designated as either safe or contaminated 31. Next the food commodity store is marked as being safe or contaminated 33. A further step illustrated in these embodiments involves tracking the marked food commodity store 35 in order to ensure that the material is safe once it arrives at the next destination in the food commodity transport system.

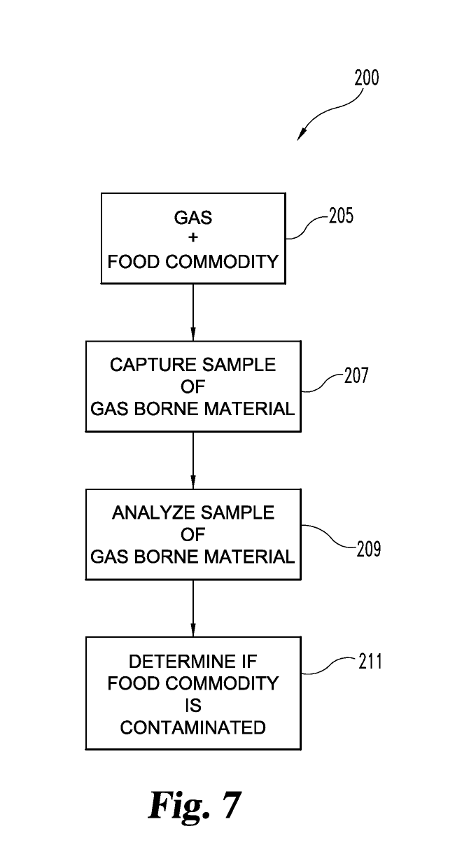

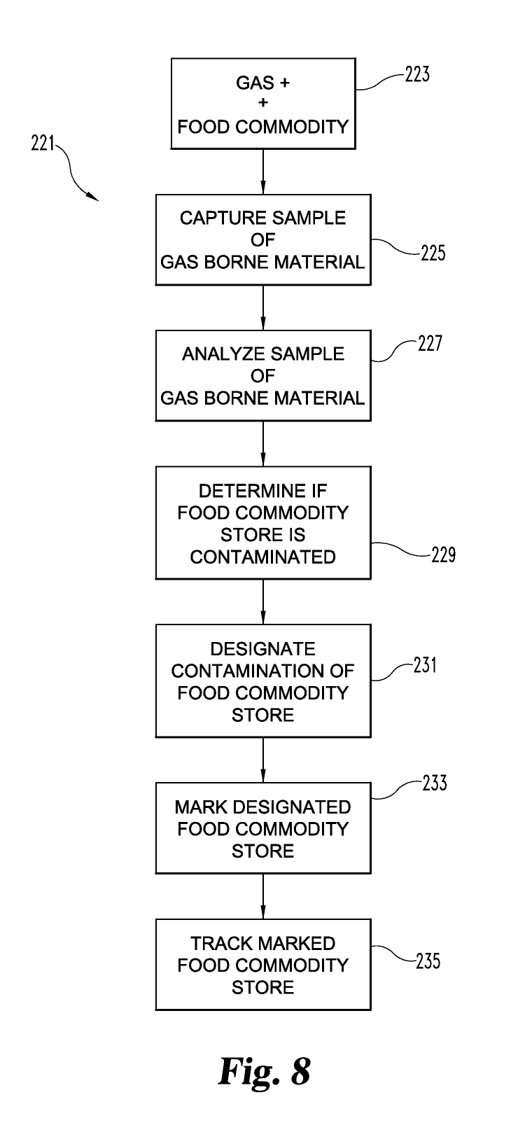

FIGS. 7 and 8 are the same, respectively, as FIGS. 1 and 2 described above, except that food commodity has be substituted for grain, and the reference characters have been modified to have a “2” in the hundreds digit for the corresponding feature.

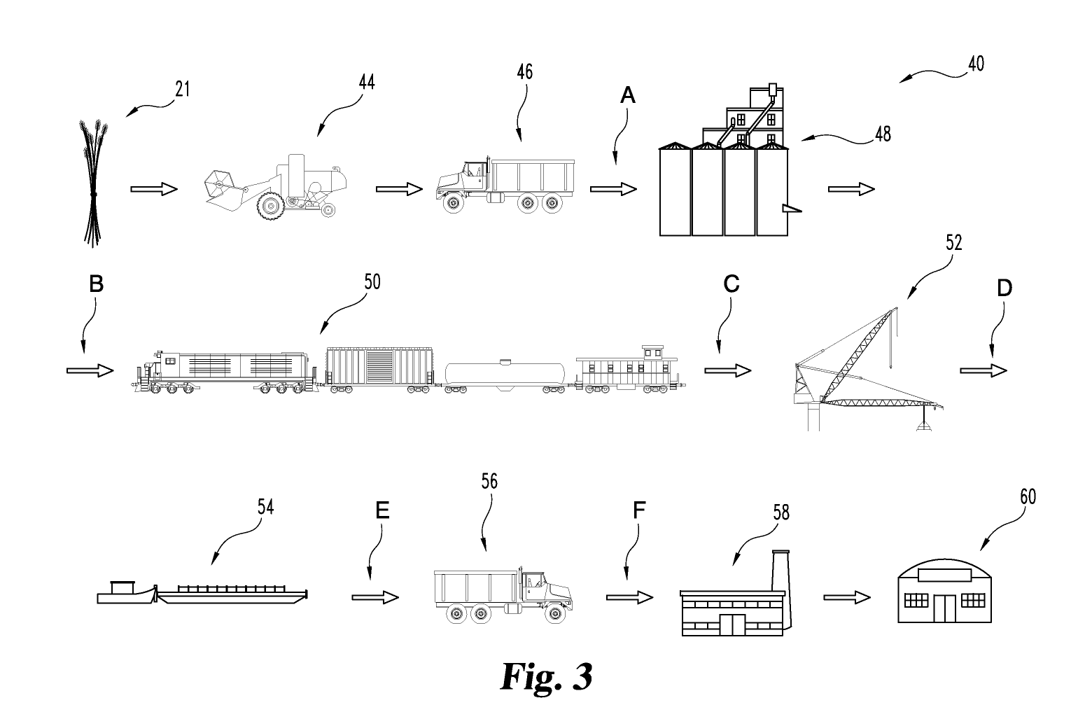

Referring now to FIG. 3, illustrated herein are various steps commonly found in the process of growing, harvesting, and transporting food commodity to the retail market 40. Various steps include harvesting, for example, wheat 21 using a mechanized device 44, for example, a combine. Next, harvested food commodity is loaded onto a truck 46 for delivery to a grain storage facility, for example, a food commodity elevator 48. Next, material food commodity stores accumulated in the elevator are loaded onto a train including, for example, railroad cars suitable for the containment of food commodity 50. Next, the food commodity may be delivered to a dock for eventual loading 52 onto a barge or ship or other type of container vessel 54. After transport via ship or barge, a food commodity may be offloaded onto another form of transportation, for example, a truck 56 which is used to deliver the food commodity to a food processing plant 58. Ultimately, the grain is incorporated into a food-stuff, for example, bread, for delivery to a retail market 60. Various places that can be sampled to check for contamination include batch stores, storage tanks, elevators and the like.

In still another embodiment a sample is collected from a bulk food commodity store such as the hold of ship and placed into a drum or other confined space. The food commodity in the drum may be aerated or mixed to produce a gas, for example, air that has been in contact with the food commodity and now carries a portion of the material that is the food commodity or is mixed in with the food commodity in the drum. This technique may be useful for sampling settled food commodity stores in that grab samples can be taken from various positions within the store that are not in contact with bases in the headspace of the food commodity container. It is an especially useful approach when gases in the headspace of food commodity storage are not expected to include solid material that is representative of the material in the bulk food commodity store. In one embodiment rather than testing each grab sample individually multiple grab samples may be combined to form a composite sample and the composite may be tested for the presence of contaminants including GMOs.

Further as illustrated in FIG. 3 there are various points in the food commodity harvesting and transportation chain where it may be particularly advantageous to sample the food commodity. Early on in the process, for example, at step A, it may be useful to sample each load of food commodity from individual trucks to ensure that the food commodity in each truck is safe before it is mixed in with the bulk material in the food commodity elevator 48. Catching a contaminant at this level could be particularly useful in that it would prevent a large amount of food commodity from becoming contaminated. Another point at which the grain supply can be checked for contaminants is B when the grain from the food commodity elevator is off-loaded to another medium for further delivery, such as by railroad car. Yet another place where the grain can be sampled is when it is delivered to or from a loading dock C. Sampling at this stage is important as the food commodity is being agglomerated, and inadvertently mixing in a portion of contaminated food commodity may contaminate a large store of food commodity. The contents of perhaps an entire train load of train cars collected from a number of different food commodity elevator stores for delivery to, for example, a ship or a barge may be sampled and tested. Material on the ship or barge may ultimately be offloaded at yet another dock and still another port, presenting still another sampling opportunity E. Just before delivery F of the food commodity to its processing plant is still another opportunity to sample the food commodity. Sampling at F may be a particularly important step in that food commodity at this stage is destined for immediate use as a food product for human or animal consumption. Accordingly, this is a particularly valuable place in the transfer chain to test the food commodity and ensure that the food commodity is safe for human consumption. Ultimately, assuming that the food commodity has been adequately tested through collection and delivery steps 21 through 56, the burden for ensuring the safety of the food-stuff now shifts to the food processing plant 58.

Other steps in the food commodity handling process where representative samples can be gathered include sampling gases, especially air, in and around conveyors, augurs, combines, grain dryers and the like.

Various aspects provide novel and useful ways for sampling and testing the food commodity as well as for marking and tracking, and thereby ensuring the safety of the food commodity at various steps along the pathway of producing the food commodity and ultimately converting it into a food-stuff.

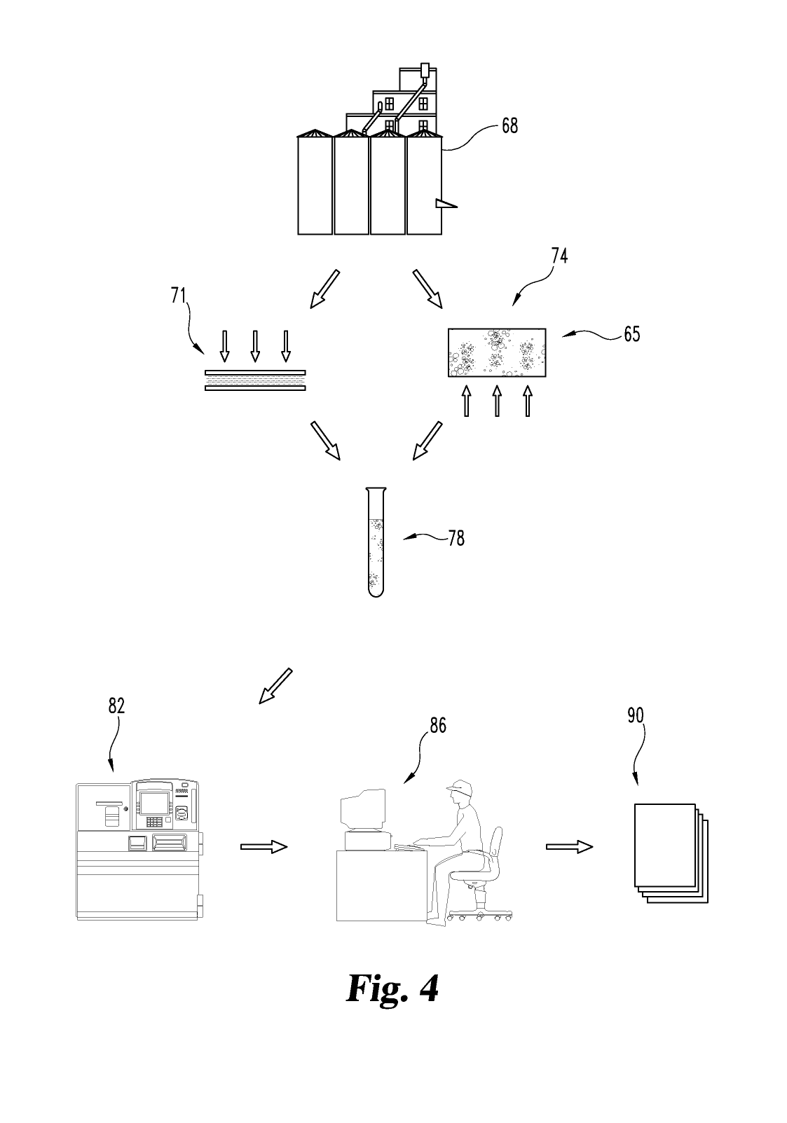

Referring now to FIG. 4, illustrated herein is a schematic diagram 65 of various ways in which a particular food commodity store, for example, food commodity in a food commodity elevator 68, may be sampled and analyzed to determine if it is contaminated. As shown herein, a capture media in the form of a filter 71 or an electrostatic filter 64 or, not illustrated but just as easily implemented, a sparger may be used to collect a sample of matter associated with gases that are in contact with the food commodity store. Samples from any of these capture media may be prepared and placed into a suitable form 78 for analysis using some type of chemical, physical, electrochemical, electro physical, or biological assay. In FIG. 4, equipment for assaying samples is illustrated as a piece of equipment 82. The next step in the process of testing the food commodity for contaminates is to produce test data perhaps in the form of a numeric output fed to a computer shown as 86 which ultimately generates a report 90. The results of these analyses and the subsequent review of the report can be used to make decisions as to whether the food commodity is, or is not, contaminated and whether it should be marked as uncontaminated or contaminated. In one aspect, food commodity tested for contamination is also marked at this stage for tracking throughout the rest of the food commodity processing steps as illustrated previously in FIGS. 1 and 2.

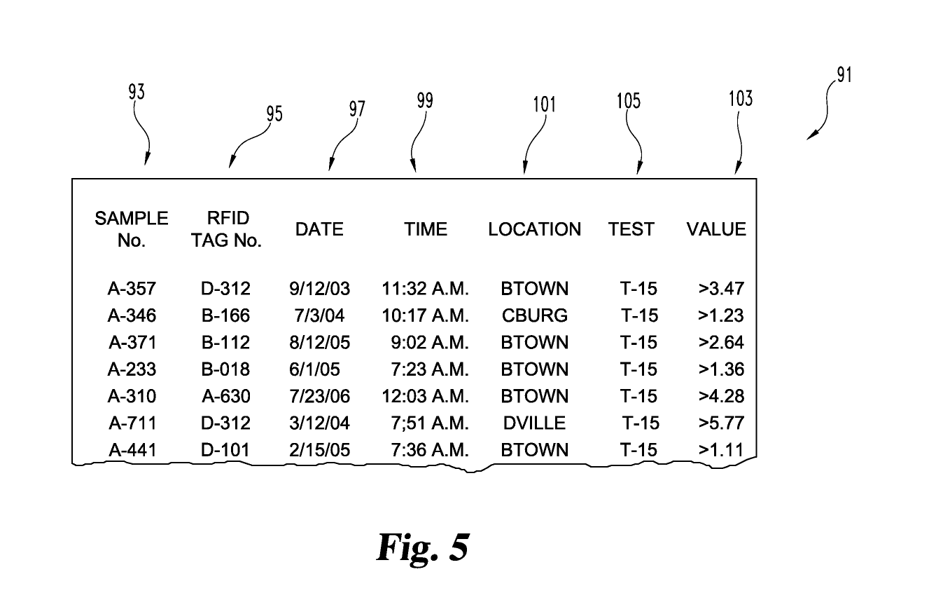

Referring now to FIG. 5, illustrating a typical report 91 as may be generated by testing and tabulating data collected from testing grain stores. Typically data in report 91 may include, for example, sample number 93, an RFID 95 tag number, which may indicate information about which capture media was used, when and where the sample was drawn, and the like. Additional information in table 92 may include the date 97 and time 99 on which the sample was collected and the location 101 where the sample was collected. Location data 101 can include, for example, information such as the following: the name of the city, town, plant, storage bin, railroad car, truck, barge, ship, elevator, and the like. Table 91 may also include a column designating the type of test performed on a given sample 105 and the value measured 103 using a given test 105.

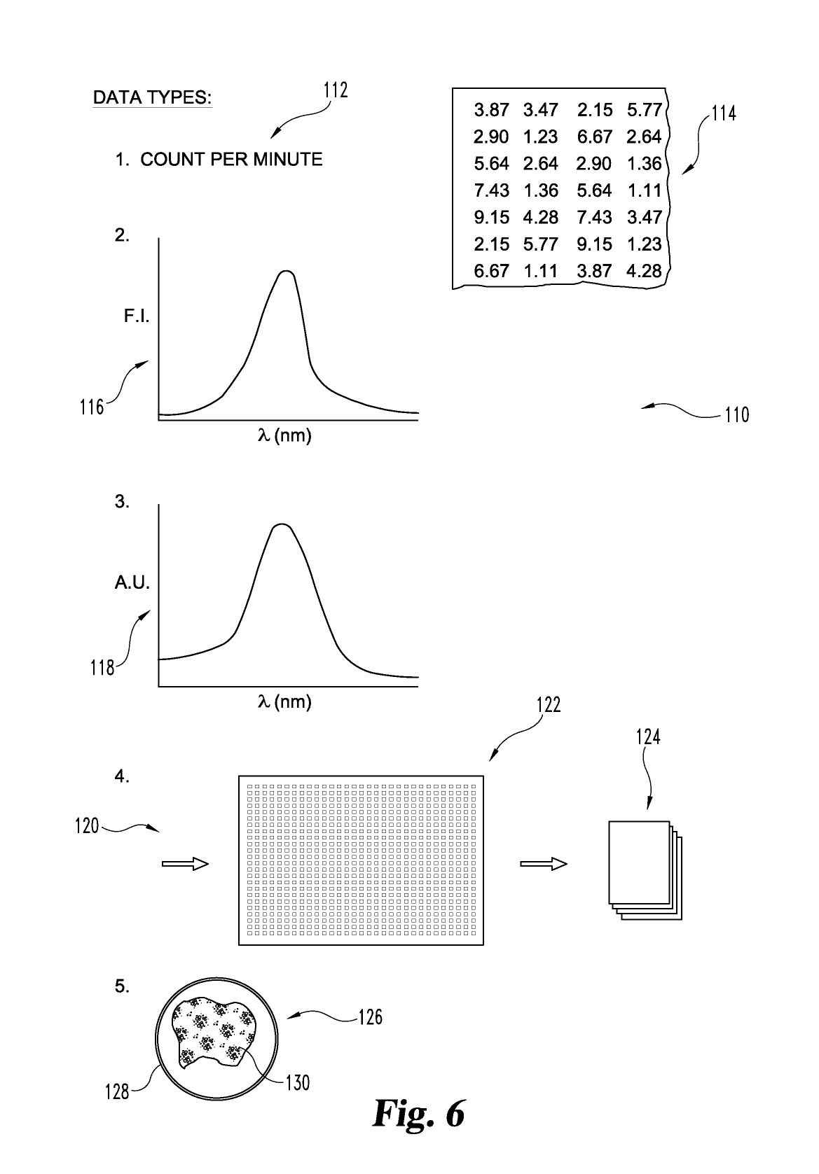

Referring now to FIG. 6, illustrated here 114 are various analytical tools that can be used to test particulates from gas associated with a food commodity store to determine if the food commodity store is contaminated. These tests include, but are not limited to, for example, counts per minute (1) which is commonly used in order to determine whether or not a radioactive contaminant is present in a particular grain store. Tests for radioactive contaminants often produce numerical values which are best presented in tabular form 114. Tests for radioactive materials may be accomplished by use of a Geiger counter, scintillation counter, or similar equipment.

Similarly, particulate samples may be tested directly or processed and then tested to determine if there is a florescent compound or signal 116 which is indicative the presence of a contaminant in the sample. Similarly, uv-absorbance 118 may be used to measure contamination levels in a given sample. Alternatively, material in a sample may be tested for contamination using an array 120; as shown herein 122 may be an array or stack of arrays 124. Arrays can include anything from nucleotide fragments which are known to hybridize complimentary nucleic acid sequences, to reagents that react with certain chemical contaminants. In one embodiment the array is comprised of a series of antibodies which selectively bind to specific antigens which are known to be, or thought to, associated with various contaminants.

One embodiment includes using the Elisa Kit to provide results on biological contaminants within a few hours. Some tests for chemical contamination generate results available in a day or less on all incoming shipments of food commodity. In one embodiment, any positive results are laboratory confirmed by follow-up testing within 72 hours.

Still another type of testing illustrated in 126 includes plating a sample of the particulate material recovered from gas in contact with the food commodity store on a plate 128. Some embodiments include using growth medium that selects for the growth of specific types of pathogenic microorganisms 130 which may be contaminating the food commodity. Evidence of growth in a specific set of plate conditions can be correlated with the presence of a contaminant of biological nature in a given food commodity store.

Ultimately, any one of these various analytical steps and many not shown can be used to generate a report which will enable the operator to make a determination as to whether or not a particular food commodity store is contaminated.

Additional testing for biological contaminant can be performed by blacklight and chromatography if cross qualitative analysis is required.

One embodiment includes forming a composite sample. The composite is a batch consisting of, for example, five to ten pounds of food commodity per total composite. The samples can be collected remotely by an intrusive device designed to penetrate a food commodity store to a desired location, then opened and used to remove a sample from the desired location. The samples can be visually examined and stored in a collection device (‘40’ gallon poly drum) for head space air collection and specific analysis as described in the above section. The amount of material collected in this manner may increase the statistical confidence in the sample by producing a sample that is representative of the bulk food commodity store. The sample may be aerated to increase the amount of particulate material in the head space associated with the sample. This approach can be used to produce a profile of food commodity stored in a bulk storage environment, eliminating the potential for statistical deviation created by drawing a single sample from a large food commodity store. As such, 90 to 99 percent confidence testing can be achieved through collecting a statistically appropriate amount of food commodity from the appropriate amount of locations based on the food commodity store.

One aspect includes visually marking an agricultural commodity before or after a processing step. In one embodiment, the markers are colored plant protein-derived pellets that can be made in different sizes and shapes for mixing into various agricultural commodities as a visual label or “taggant” during storage and shipment. The embodiments may greatly simplify the tracking and identification of commodity products in storage or in transit.

One embodiment includes a method and/or system for tracking transported food commodity. A radio-frequency identification (RFID) tag is provided for deposit in a container along with the food commodity. In one embodiment, the tag is dimensioned to approximate a size of an individual food commodity, and comprises a memory and RF communication channel. See, for example, U.S. Pat. No. 7,047,103. The RFID tag may further comprise data stored in the memory, the data including, for example, a time when the RFID tag was handled along with surrounding food commodity, information sufficient to determine a location of handling at the time the RFID tag was handled with the surrounding food commodity, and the purity of the food commodity store.

One embodiment includes a physical marker tracing caplet that possesses physical and chemical properties similar to native food commodity or other bulk products, does not segregate during handling, and can contain bar coding that identifies food commodity and other products from an individual field or location. One embodiment utilizes scanning technology and a data retrieval and management system that can trace food commodity and product movement and is scalable to handle the entire U.S. grain marketing system. The confidential database will also provide information on any agri-chemicals applied during production, handling or storage.

Various embodiments may include methods of identifying the source of food commodity (field level) at any point in the marketing system. The methods include marking the food commodity store by, for example, the addition of tracking beads that are readily removed from 2 kg grain sample using laboratory food commodity cleaning equipment to identify food commodity origins in a bulk shipment, have printed codes that identify the specific field of origin, contain a translucent coat to protect the printed codes during handling, and are added to the food commodity on a scale sensitive amount to provide statistical confidence that food commodity from an individual field is contained or not contained within a bulk of shipment of food commodity. Another embodiment includes adding colored plant protein-derived pellets to designated tested stores of food commodity. See, for example, U.S. Pat. No. 6,406,728 B1.

### EXAMPLE 1

Particulate samples will be collected for analysis using a dust collection system at two points, which will be chosen based on the design of the food commodity processing facility. These samples will be collected using air pumps connected to particulate filters. The pumps will be calibrated such that the equivalent amount of air from the elevator is pushed through the particulate collector for each sampling event. The particulate filter will be changed out each hour of operation for sampling for contaminants of concern.

The filter will be changed hourly for sampling. The filter will be placed into a separate container for transport to an area set aside for field sampling. A background sample will be collected by running the particulate filter in the elevator when it is not running and/or by taking particulate samples for the outside air in the vicinity of the food commodity elevator. These background samples will be analyzed in the same fashion as the samples collected in the elevator.

What is claimed:

1. A method for checking the safety of a food commodity, comprising the acts of:
(a) providing a bulk quantity of food commodity after harvesting and before processing into a final food product;
(b) passing at least a sample of gas that was in contact with a material in the bulk food commodity through a capturing media by means of positive or negative pressure;
(c) extracting or removing at least some of said material from said capturing media; and,
(d) testing said material for the presence of a microbiological contaminant.
